(19) United States
(12) Patent Application Publication
(10) Pub. No.: US 2019/0328694 A1
(43) Pub. Date: Oct. 31, 2019

(54) COMPOSITION FOR PROMOTING SKELETAL MUSCLE ACTIVITY VIA INDUCTION OF MITOCHONDRIAL BIOGENESIS COMPRISING OF AZELAIC ACID AS AN ACTIVE INGREDIENT

(71) Applicant: Korea University Research and Business Foundation, Seoul (KR)

(72) Inventor: Sung-Joon LEE, Seoul (KR)

(73) Assignee: Korea University Research and Business Foundation, Seoul (KR)

(21) Appl. No.: 16/278,899

(22) Filed: Feb. 19, 2019

(30) Foreign Application Priority Data
Feb. 20, 2018 (KR) 10-2018-0020070

(51) Int. Cl. A61K 31/20; A23L 33/10; A61P 21/00

(52) U.S. Cl. CPC A61K 31/20; A61P 21/00; A23L 33/10

(57) ABSTRACT

The present invention relates to a composition for promoting mitochondrial biogenesis, which includes azelaic acid as an active ingredient. It was confirmed that azelaic acid is a compound mainly contained in cereals and natural products and has the advantage of no or little side effects, and mitochondrial autophagy and mitochondrial DNA synthesis in cells are induced, thereby ultimately having activity of increasing mitochondrial density. Therefore, azelaic acid can be used for the treatment of mitochondrial dysfunction-associated disease by the failure of homeostasis control of the mitochondria, for example, mitochondrial activity or the decrease in the number of mitochondria. In addition, azelaic acid can be provided to reinforce a muscle function and prevent muscle aging using the activity, and to be effectively used in food material, a pharmaceutical composition, and health functional foods for treating mitochondrial dysfunction-associated disease, reinforcing a muscle function, or preventing muscle aging.

### CROSS-REFERENCE TO RELATED APPLICATION

This application claims priority to and the benefit of Korean Patent Application No. 10-2018-0020070, filed on Feb. 20, 2018, the disclosure of which is incorporated herein by reference in its entirety.

### TECHNICAL FIELD

The present invention relates to a composition for promoting mitochondrial biogenesis and composition for preventing, treating or improving a mitochondrial dysfunction-associated disease, each of which includes azelaic acid as an active ingredient.

### BACKGROUND ART

Olfactory perception means that odorants bind to olfactory receptors (ORs) and are perceived through a signaling pathway. ORs, the largest protein family of G-protein coupled receptors (GPCRs), are proteins present in about 400 types in a human and involved in olfactory perception through a signaling pathway by binding to odorants in the olfactory epithelium. A recent study has reported that ORs are ectopically expressed in various tissues of a kidney, a liver, a small intestine, etc., as well as olfactory cells, but this is the early stage such that only a few OR functions are known.

Studies on biological efficacy of compounds included in foods and natural substances have primarily focused on non-volatile materials extracted with a polar/non-polar solvent, and studies on the biological functions of aromatic fragrance components, which are derived from volatile substances, are very limited except for some aromatherapy-related studies. According to the trends of recent studies, it has been reported that aromatic essential oils isolated from herbs such as the genus Allium have a strong antioxidant activity and are effective in controlling a lipid, blood sugar and a body fat (Wenwick G R et al.), but the molecular target of a single fragrance component has been little known.

The chemical name of azelaic acid is nonanedioic acid, which is a dicarboxylic acid having 9 carbon atoms. Azelaic acid is produced in the body through an omega-oxidation process, or as peroxide of linoleic acid, or ingested since it is a natural substance present in various cereals such as wheat, barley, oatmeal, sorghum, etc., and cranberries. Azelaic acid is naturally produced in the human body through fatty acid omega-oxidation. According to recent studies, it has been known that azelaic acid is effective against inflammatory dermal diseases such as redness, acne, etc., and some studies on atherosclerosis and anticancer activity have been reported, but detailed mechanisms such as the identification of a target protein are not studied yet.

Recently, as it is known through various studies that mitochondrial dysfunction affects senescence as well as adult diseases such as diabetes and hypertension, and chronic diseases such as Parkinson's disease and dementia, the importance of the maintenance of mitochondrial function is increasing.

The mitochondrion is an organelle which primarily produces ATP, which is an energy source in cells, and has a variety of functions in cells such as metabolism, signal transduction, apoptosis, differentiation, etc. Mitochondria have mitochondrial DNA (mtDNA) which is distinguished from a cell's own nuclear DNA. mtDNA does not have a repair mechanism for repairing damage unlike nuclear DNA of a cell, and since there is no histone protein serving to protecting DNA, it is relatively prone to damage. mtDNA damage leads to mitochondrial dysfunction, a decrease in synthesis of ATP which is an energy source required for cell activity, and a decrease in ability to regulate homeostasis in the body, resulting in the onset of various diseases.

Endurance exercises such as running, swimming, etc. raise skeletal muscle respiratory capacity, which affects the increase of enzymes involved in the electron transport chain and the citric acid cycle of mitochondria, and fatty acid oxidation. In addition, the size and number of mitochondria in skeletal muscle are increased by increasing the rate of synthetic metabolism rather than the degradation metabolism of mitochondrial proteins. Therefore, since the improvement in oxygen utilization ability in skeletal muscle mitochondria improves ATP production capacity through oxidative phosphorylation, studies on mitochondrial biogenesis in skeletal muscle are very important in the research on improving exercise capacity.

The metabolic adaptation in skeletal muscle is accomplished by regulating various genes, and the most critical factor of this response is a peroxisome proliferator-activated receptor-γ coactivator-1α (PGC-1α) transcription cofactor. PGC-1α plays a pivotal role in energy metabolism and mitochondrial biogenesis by being activated by upstream signaling mechanisms such as Ca²⁺-regulated CAMKIV-calcineurin/NFAT and the MEF2 axis, adrenergic/cholinergic signaling, and AMP-activated protein kinase (AMPK) during exercise. In addition, PGC-1α has been known to regulate the expression of genes involved in sugar metabolism such as glucose transporter type 4, which is a blood sugar transporter, and pyruvate dehydrogenase kinase 4 (PDK4) inhibiting oxidation, as well as mitochondrial biogenesis.

### DISCLOSURE

### Technical Problem

The inventors confirmed that Olfr544 is expressed in muscle cells, azelaic acid acts as a ligand of Olfr544 in muscle cells to activate a CREB-PCG-1α pathway and an ERK1/2 signaling pathway, thereby inducing the autophagy of mitochondria, and increasing mtDNA and mitochondrial density, and thus the present invention was completed.

Therefore, the present invention is directed to providing a composition for promoting mitochondrial biogenesis, which includes azelaic acid as an active ingredient.

The present invention is also directed to providing a composition for preventing, treating or improving a mitochondrial dysfunction-associated disease, which includes azelaic acid as an active ingredient.

The present invention is also directed to providing a composition for reinforcing muscle function or preventing muscle aging, which includes azelaic acid as an active ingredient.

However, technical problems to be solved in the present invention are not limited to the above-described problems, and other problems which are not described herein will be fully understood by those of ordinary skill in the art from the following descriptions.

### Technical Solution

To solve the above-described technical problems, the present invention provides a composition for promoting mitochondrial biogenesis, which includes azelaic acid as an active ingredient.

In an exemplary embodiment of the present invention, the composition may activate Olfr544.

In another exemplary embodiment of the present invention, the composition may increase the expression of PGC-1α.

In still another exemplary embodiment of the present invention, the composition may activate ERK1/2.

In addition, the present invention provides a pharmaceutical composition for preventing or treating a mitochondrial dysfunction-associated disease, which includes azelaic acid as an active ingredient.

The present invention provides a food composition for preventing or improving a mitochondrial dysfunction-associated disease, which includes azelaic acid as an active ingredient.

The present invention provides a composition for reinforcing a muscle function or preventing muscle aging, which includes azelaic acid as an active ingredient.

In an exemplary embodiment of the present invention, the composition may reinforce a muscle function and prevent muscle aging through the activation of mitochondrial function.

In another exemplary embodiment of the present invention, the composition may induce the autophagy of mitochondria in a cell.

In still another exemplary embodiment of the present invention, the composition may promote mitochondrial biogenesis in a cell.

In yet another exemplary embodiment of the present invention, the induction of the autophagy and/or promotion of biogenesis of the mitochondria may be caused by Olfr544 and ERK1/2 activation.

In addition, the present invention provides a method of preventing or treating a mitochondrial dysfunction-associated disease, which includes administering azelaic acid to a subject.

In addition, the present invention provides a use of azelaic acid for preparing a drug for preventing or treating mitochondrial dysfunction-associated disease.

In addition, the present invention provides a method of reinforcing a muscle function or preventing muscle aging, which includes administering azelaic acid or a pharmaceutically acceptable salt thereof to a subject.

In addition, the present invention provides a method of treating a mitochondrial dysfunction-associated disease, which includes administering azelaic acid or a pharmaceutically acceptable salt thereof to a subject.

In addition, the present invention provides a use of azelaic acid for preparing a drug for reinforcing a muscle function or preventing muscle aging.

In addition, the present invention provides a use of azelaic acid for preparing a drug for treating mitochondrial dysfunction-associated disease.

### Advantageous Effects

It was confirmed that azelaic acid according to the present invention is a compound mainly contained in cereals such as wheat, oats, barley, sorghum and natural products such as cranberries, etc. and has the advantage of no or little side effects, and mitochondrial autophagy and mitochondrial DNA synthesis in cells are induced, thereby ultimately having activity of increasing mitochondrial density. Therefore, azelaic acid can be used for the treatment of mitochondrial dysfunction-associated disease due the failure of homeostasis control of the mitochondria, for example, mitochondrial activity or the decrease in the number of mitochondria. In addition, azelaic acid can be provided to reinforce a muscle function and prevent muscle aging using the activity, and to be effectively used in food material, a pharmaceutical composition, and health functional foods for treating mitochondrial dysfunction-associated disease, reinforcing a muscle function, or preventing muscle aging.

### DESCRIPTION OF DRAWINGS

The above and other objects, features and advantages of the present invention will become more apparent to those of ordinary skill in the art by describing in detail exemplary embodiments thereof with reference to the accompanying drawings, in which:

FIG. 1 is a diagram evaluating the cytotoxicity of azelaic acid using an MTT assay.

FIG. 2A and FIG. 2B are images for confirming the expression of an Olfr544 gene in muscle cells (FIG. 2A: confirmation of Olfr544 and Olfr545 plasmid expression, FIG. 2B: confirmation of Olfr544 and Olfr545 expression in myotubes, myocytes and muscle).

FIG. 3A to FIG. 3E are graphs showing a mitochondrial biogenesis promotion effect of azelaic acid (FIG. 3A: analysis of PGC-1α gene expression according to azelaic acid, FIG. 3B: analysis of PGC-1α protein expression according to azelaic acid, FIG. 3C: analysis of mtDNA expression level according to azelaic acid, FIG. 3D: analysis of the change in mitochondrial density according to azelaic acid, and FIG. 3E: density change according to azelaic acid, measured by immunofluorescence-based imaging).

FIG. 4A to FIG. 4E are images showing the Olfr544-dependent mitochondrial biogenesis effect of azelaic acid (FIG. 4A and FIG. 4B: confirmation of Olfr544 knockdown, FIG. 4C: analysis of PGC-1α protein expression, FIG. 4D: analysis of mtDNA expression level, and FIG. 4E: analysis of mitochondrial density, in Olfr544 knockdown cells and normal cells).

FIG. 5A to FIG. 5F are diagrams showing mechanisms of promoting mitochondrial biogenesis according to Olfr544 activity of azelaic acid (FIG. 5A and FIG. 5B: the analysis result of pCREB and CREB protein expression, FIG. 5C and FIG. 5D: the analysis result of pERK1/2 and ERK1/2 protein expression, FIG. 5E and FIG. 5F: the analysis result of LC3 protein expression, according to azelaic acid treatment in Olfr544 knockdown cells and normal cells).

FIG. 6A to FIG. 6C are graphs showing an effect of increasing mitochondrial biogenesis according to the administration of azelaic acid to mice (FIG. 6A: analysis of PGC-1α gene expression level, FIG. 6B: analysis of Tfam gene expression level, and FIG. 6C: analysis of mtDNA expression level, when Olfr544 KO cells and normal cells are treated with azelaic acid).

FIG. 7A to FIG. 7E are diagrams showing mechanisms of promoting mitochondrial biogenesis according to Olfr544 activity of azelaic acid in mice (FIG. 7A: the images showing pCREB, CREB, PGC-1α, ERK1/2, pERK1/2 and LC3 protein expression, FIG. 7B: the analysis of pCREB and CREB protein expression levels, FIG. 7C: the analysis of PGC-1α protein expression per azelaic acid treatment time, FIG. 7D: the analysis of pERK and ERK protein expression levels, and FIG. 7E: the analysis of LC3 protein expression by per azelaic acid treatment time in muscle tissue extracted 30 and 120 minutes after azelaic acid is intraperitoneally injected into normal mice and Olfr544 KO mice).

### MODES OF THE INVENTION

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

The inventors confirmed that azelaic acid mainly contained in cereals such as wheat, oats, barley and sorghum and natural products such as cranberries, etc. increases mitochondrial biogenesis in muscle cells, and through a further study, confirmed that azelaic acid functions as a ligand of Olfr544, which is a G-protein coupled receptor (GPCR), in muscle cells to activate an Olfr544-cAMP response element binding protein (CREB)-PGC-1α pathway, and promotes mitochondrial biogenesis in cells by activating an ERK1/2 signaling pathway, and thus the present invention was completed.

The term “azelaic acid (AzA)” used herein refers to nonanedioic acid, and has the structure of Formula 1, which has a molecular weight of 188.22 g/mol and a molecular formula of C₉H₁₆O₄.

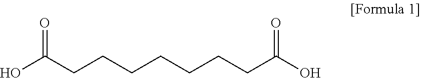 [SMILES: O=C(O)CCCCCCCC(=O)O]

Since the azelaic acid is a compound mainly contained in cereals such as wheat, oats, barley and sorghum and natural products such as cranberries, etc. and has been known to be produced by omega oxidation of fatty acids in the human body, the azelaic acid has an advantage of no or little side effects when administered to a subject. It was demonstrated that azelaic acid present in the human body is safe for ingestion thereof at a concentration of about 10 to 50 μM. In one exemplary embodiment of the present invention, as a result of evaluating the cytotoxicity of azelaic acid through an MTT assay, it was observed that, even when a high concentration of azelaic acid is treated, there is no change in cell viability, reconfirming the safety of azelaic acid (see Example 1).

Since the azelaic acid of the present invention is a natural substance and thus is not toxic, it can be continuously used in large quantities as an active ingredient for food or medicine.

The azelaic acid of the present invention may be obtained from cereals such as wheat, oats, barley and sorghum and natural products such as cranberries, etc. by a conventional extraction method such as juice extraction, vapor extraction, hot water extraction, ultrasonic extraction, solvent extraction or reflux cooling extraction, and for the extraction, one or more solvents selected from the group consisting of water, an alcohol having 1 to 4 carbon atoms, n-hexane, ethyl acetate, acetone, butyl acetate, 1,3-butylene glycol, methylene chloride, and a mixed solvent thereof may be used, but the present invention is not limited thereto.

In addition, the azelaic acid of the present invention may be a synthetic compound, but it is obvious that the azelaic acid can have the same efficacy as obtained from the natural product and can be used for the same purpose as that obtained from the natural product.

The inventors confirmed the efficacy of promoting mitochondrial biogenesis by azelaic acid acting as a ligand of an olfactory receptor ectopically expressed in muscle cells. First, they confirmed olfactory receptor Olfr544 expression in muscle cells, and an increase in mitochondrial biogenesis-related markers according to the treatment of muscle cells with azelaic acid. Afterward, Olfr544 expression was artificially reduced using Olfr544 siRNA-treated cells and an Olfr544 knockout mouse, and compared to the control group, it was confirmed whether the mitochondrial biogenesis efficacy of azelaic acid is exhibited in an Olfr544-dependent manner.

More specifically, the inventors confirmed Olfr544 expression in muscle cells (see Example 2), and also confirmed that mtDNA and mitochondrial density are increased according to the treatment of muscle cells with azelaic acid, and an increase in expression of PGC-1α regulating mitochondrial functions (see Example 3).

In addition, the inventors confirmed that, by the treatment of Olfr544 knockdown myocytes in which Olfr 544 expression is reduced by transforming Olfr544 siRNA and normal cells with azelaic acid, PGC-1α expression, mtDNA and mitochondrial density are increased in normal cells, but not in Olfr544 knockdown cells (see Example 4), suggesting that azelaic acid serves as a ligand of Olfr544 in myocytes to induce Olfr544-dependent mitochondrial biogenesis.

Meanwhile, PGC-1α is a key element of mitochondrial function, and considered a master regulator of mitochondrial biogenesis and a strong coactivator of the overactivation of a transcription factor affecting fatty acid oxidation and energy consumption in the skeletal muscle in the entire body. In addition, PGC-1α is a coactivator of nuclear transcription factors such as nuclear respiratory factor-1 (NRF-1) and transcription factor A (TFAM), which are necessary for mitochondrial gene expression and genome replication.

In addition, the inventors confirmed the change in expression levels of CREB/pCREB, EKR1/2 and LC3II/LC3I by treating Olfr544 siRNA-transfected Olfr544 knockdown cells and normal cells with azelaic acid to more specifically investigate a mitochondrial biogenesis mechanism of azelaic acid in myocytes, demonstrating that, unlike the Olfr544 knockdown cells, pCREB, pERK1/2 and LC3II/LC3I expression according to the treatment of normal cells with azelaic acid is increased, and thus it can be seen that azelaic acid promotes CREB-PGC-1α signaling and ERK1/2 activation in skeletal muscle tissue, and induces autophagy in the skeletal muscle cells (see Example 5).

Meanwhile, mitophagy refers to mitochondrial autophagy, and mitochondrial degradation by autophagy is a key mechanism of regulating mitochondrial homeostasis as well as mitochondrial biogenesis. Here, it has been known that autophagy prevents damage to mitochondria in skeletal muscle cells, improves the adaptability of muscle tissue during exercise, and plays a critical role in mitochondrial biogenesis. That is, suitable autophagy is a key mechanism which can inhibit aging of myocytes so they can smoothly perform their function.

Accordingly, the azelaic acid of the present invention may induce mitochondrial autophagy in myocytes to prevent damage to mitochondria in skeletal muscle cells, improve muscle performance, increase a tissue ATP level, and promote mitochondrial biogenesis during exercise.

Therefore, the azelaic acid of the present invention may be used in prevention, treatment or improvement of mitochondrial dysfunction-associated disease.

The term “mitochondrial dysfunction-associated disease” used herein refers to various types of degenerative diseases, brain diseases, neurological diseases, heart diseases, liver diseases, kidney diseases, pancreatic diseases, metabolic diseases or muscle diseases, caused by the failure of regulation of mitochondrial homeostasis such as a decrease in mitochondrial activity, a decrease in mitochondrial function, inappropriate mitochondrial activity, or a decrease in number of mitochondria. The degenerative disease may be degenerative arthritis, rheumatoid arthritis or osteoarthritis, the brain disease may be dementia, Parkinson's disease, stroke, developmental retardation, a neuropsychiatric disorder, a migraine, autism, mental retardation, seizures or stroke, the neurological disease may be ptosis, optic atrophy, strabismus, retinitis pigmentosa, blindness, hearing loss, eye muscle paralysis, hyporeflexia, syncope, nerve pain or autonomic imbalance, the heart disease may be a heart attack or cardiomyopathy, the liver disease may be hypoglycemia or hepatic insufficiency, the kidney disease may be nephrocalcinosis, the pancreatic disease may be pancreatic exocrine insufficiency or hypoparathyroidism, the metabolic disease may be hypertension, diabetes or obesity, and the muscle disease may be irritable bowel syndrome, muscular pain, muscular dystrophy, gastroesophageal reflux disease, hypotension, a convulsion or a motor disturbance, but the present invention is not limited thereto.

The composition of the present invention regulates mitochondrial homeostasis in cells, thereby promoting neuroprotective, neurotrophic, and/or neurite proliferation, and ultimately improving cognitive function.

In addition, the composition of the present invention regulates mitochondrial homeostasis in cells to increase a metabolic rate, reduce a fat ratio, increase muscle mass, inhibit body weight gain or induce the decrease in body weight, improve mental ability (including memory), maintain the improvement in muscle performance, improve moods or manage stress, and thus can be effective in maintenance of the body and mental health of a subject.

According to the above, it can be seen that the azelaic acid of the present invention can reinforce muscle function and delay muscle aging through mitochondrial activation. The present invention provides a composition for reinforcing muscle function or preventing muscle aging, which includes azelaic acid as an active ingredient, and includes, but is not limited to, a pharmaceutical composition, a plant composition, and a health functional food composition.

The term “muscle function” used herein refers to performing a function of maintaining a force for performing an operation, a force for maintaining posture, or generating heat for body temperature maintenance by contraction, elasticity, excitability and conductivity of muscle fibers that constitute muscle.

In addition, the term “muscle aging” used herein refers to sarcopenia, in which muscle function is weakened as mitochondria in muscle fibers lose their activity, or the number of mitochondria decreases.

In an exemplary embodiment of the present invention, the inventors induced obesity in an Olfr544 knockout mouse and a normal mouse with a high-fat diet (HFD) and who were then orally administered azelaic acid, thereby confirming that PGC-1α and Tfa expression increased in the normal mouse due to the administration of azelaic acid, and thus mtDNA increased. In addition, it was confirmed that, when azelaic acid was intraperitoneally administered, compared to the control group, pERK and LC3II/LC3I expression increased (see Example 6). Therefore, it can be seen that the composition of the present invention is effective in prevention, treatment or improvement of obesity, particularly, induced by a high-fat diet.

In addition, the composition of the present invention includes azelaic acid as an active ingredient, and further includes one or more materials conventionally used to prevent or treat mitochondrial dysfunction-associated disease.

The term “prevention” used herein refers to all actions that delay the onset of a mitochondrial dysfunction-associated disease by administration of the pharmaceutical composition according to the present invention, the term “treatment” used herein refers to all actions that alleviate or beneficially change symptoms of a mitochondrial dysfunction-associated disease by administration of the pharmaceutical composition according to the present invention, the term “improvement” used herein refers to all actions that diminish the degree of symptoms, for example, parameters associated with a mitochondrial dysfunction-associated disease, by administration of the pharmaceutical composition according to the present invention.

In the present invention, azelaic acid may be used in the form of a pharmaceutically acceptable salt, and as a salt, an acid-addition salt formed by a pharmaceutically acceptable free acid is preferable.

The term “salt” used herein is preferably an acid-addition salt formed by a pharmaceutically acceptable free acid. The acid-addition salt is obtained from an inorganic acid such as hydrochloric acid, nitric acid, phosphoric acid, sulfonic acid, hydrobromic acid, hydroiodic acid, nitrous acid or phosphorous acid, an aliphatic mono or dicarboxylate, a phenyl-substituted alkanoate, hydroxy alkanoate or alkandioate, an aromatic acid, or a non-toxic organic acid such as an aliphatic or aromatic sulfonic acid. Such pharmaceutically non-toxic salts include sulfates, pyrosulfates, bisulfates, sulfites, bisulfites, nitrates, phosphates, monohydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexane-1,6-dioates, benzoates, chlorobenzoates, methyl benzoates, dinitrobenzoates, hydroxy benzoates, methoxy benzoates, phthalates, terephthalates, benzene sulfonates, toluenesulfonates, chlorobenzene sulfonates, xylene sulfonates, phenylacetates, phenylpropionates, phenylbutyrates, citrates, lactates, β-hydroxybutyrates, glycolates, malates, titrates, methanesulfonates, propane sulfonates, naphthalene-1-sulfonates, naphthalene-2-sulfonates, or mandelates.

The acid-addition salt according to the present invention may be prepared by a conventional method, for example, dissolving a compound represented by Formula 1 in an excessive amount of an acidic aqueous solution, and precipitating the salt using a water-miscible organic solvent, for example, methanol, ethanol, acetone or acetonitrile. Alternatively, the acid-addition salt according to the present invention may be prepared by evaporating the solvent or an excessive amount of acid from the mixture and then drying the resulting product, or suction-filtering the precipitated salt.

In addition, a pharmaceutically acceptable metal salt may be prepared using a base. An alkali metal or alkaline earth metal salt is obtained, for example, by dissolving a compound in an excessive amount of an alkali metal hydroxide or alkaline earth metal hydroxide solution, filtering a non-soluble compound salt, and evaporating and drying the filtrate. Here, as a metal salt, a sodium, potassium or calcium salt is preferable. The corresponding silver salt is obtained by reacting an alkali metal or alkaline earth metal with a suitable silver salt (e.g., silver nitrate).

In addition, the compound of the present invention includes all salts, isomers, hydrates and solvates, which can be prepared by conventional methods, as well as the pharmaceutically acceptable salt.

In the present invention, the pharmaceutical composition may further include suitable carrier, excipient and diluent, which are conventionally used in the preparation of a pharmaceutical composition.

The term “carrier” used herein is also called a vehicle, and means a compound that facilitates the addition of a compound into cells or tissue. For example, dimethyl sulfoxide (DMSO) is a carrier conventionally used to facilitate the input of various organic compounds into cells or tissue of an organism.

The term “diluent” used herein is defined as a compound which not only stabilizes a biologically active form of a target compound, but also dilutes the compound in water for dissolving the compound. A salt dissolved in a buffer is used as a diluent in the art. A conventionally used buffer is phosphate buffered saline, and this is because it imitates a salt state of the human solution. Since the buffer salt can control the pH of a solution at a low concentration, the buffer diluent rarely modifies the biological activity of a compound. Compounds containing azelaic acid, which are used herein, may be administered to a human patient, or in the form of a pharmaceutical composition in combination with other components or a suitable carrier or excipient, as used in combination therapy.

In addition, the pharmaceutical composition for preventing or treating a mitochondrial dysfunction-associated disease, which includes azelaic acid according to the present invention may be used after formulation in the form of powder, a granule, a tablet, a capsule, a suspension, an emulsion, a syrup, an agent for external use, for example, an aerosol, or a sterile injection according to a conventional method, and carriers, excipients and diluents which can be included in the composition including azelaic acid may be lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. In preparation, the composition of the present invention may be formulated using a diluent or an excipient such as a filler, a thickening agent, a binder, a wetting agent, a disintegrant or a surfactant, which is conventionally used. A solid formulation for oral administration may be a tablet, pill, powder, granule or capsule, and such a solid formulation may be prepared by mixing at least one excipient, for example, starch, calcium carbonate, sucrose, lactose, and gelatin, with the active ingredient. Also, in addition to the simple excipient, lubricants such as magnesium stearate and talc may also be used. As a liquid formulation for oral administration, a. suspension, a liquid for internal use, an emulsion, or a syrup may be used, and a generally-used simple diluent such as water or liquid paraffin, as well as various types of excipients, for example, a wetting agent, a sweetener, a fragrance, and a preservative may be included. A formulation for parenteral administration includes a sterilized aqueous solution, a non-aqueous solvent, a suspension, an emulsion, a lyophilizing agent and a suppository. As the non-aqueous solvent or suspension, propylene glycol, polyethylene glycol, vegetable oil such as olive oil, or an injectable ester such as ethyl oleate may be used. As a suppository base, Witepsol, Tween 61, cacao butter, laurin fat, or glycerogelatin may be used.

A therapeutically effective amount of the compound containing azelaic acid according to the present invention may be measured at an early stage of a cell culture assay. For example, a dose may be calculated in an animal model to obtain a circulation concentration range including a half maximal inhibitory concentration (IC50) or half maximal effective concentration (EC50), determined in cell culture. This information can be used to more exactly determine a useful dose in a human. A dose of azelaic acid may be changed within the range according to an administration type employed and an administration route utilized.

A preferable dose of the pharmaceutical composition of the present invention may vary according to a patient's condition and body weight, the severity of a disease, a drug type, an administration route and duration, and may be appropriately selected by one of ordinary skill in the art. However, for a preferable effect, the pharmaceutical composition of the present invention may be administered daily at 0.0001 to 1000 mg/kg, preferably 0.5 to 200 mg/kg, and more preferably 0.5 to 100 mg/kg. Administration may be performed one or several times per day. The dose does not limit the range of the present invention in any aspect.

The pharmaceutical composition according to the present invention may be administered to mammals such as a rat, a mouse, livestock, and a human by various routes such as parenteral administration, oral administration, and the like, and a route of administration may be expected, and the pharmaceutical composition according to the present invention may be administered, for example, orally, or by intrarectal, intravenous, intramuscular, subcutaneous, intrauterine dura mater or intracerebroventricular injection.

In addition, an oral formulation may vary according to a patient's age, sex or body weight, and may be administered at 0.1 to 100 mg/kg one to several times per day. In addition, the dose may be increased/decreased according to an administration route, the degree of a disease, sex, body weight, or age. Therefore, the dose does not limit the range of the present invention in any aspect.

In the present invention, when provided as a mixture containing other components in addition to azelaic acid, the composition may include the azelaic acid at 0.001 to 99.9 wt %, preferably 0.1 to 99.0 wt %, and more preferably 30 to 50 wt % with respect to the total weight of the composition.

In addition, the present invention provides a food composition for preventing or improving a mitochondrial dysfunction-associated disease, which includes azelaic acid as an active ingredient. In addition, azelaic acid may be added to food for improving mitochondrial dysfunction-associated disease or vascular disease. When the azelaic acid of the present invention is used as a food additive, the azelaic acid may be added alone or in combination with another food or food component, and may be suitably used according to a conventional method. A mixing amount of the active ingredient may be suitably determined according to a purpose of use (prevention, health or therapeutic treatment). Generally, in manufacture of food or a drink, the azelaic acid of the present invention is added at 15 wt % or less, and preferably 10 wt % or less with respect to the raw components. However, in the case of long-term ingestion for health and hygiene or for health control, the amount may be less than the above range, and since there is no problem in terms of safety, the active ingredient may be used at an amount exceeding the above range.

In the present invention, the food includes functional food and health functional food, and the term “functional food” used herein means food improved in functionality of general food by adding the azelaic acid of the present invention to general food. The functionality may be classified into a physical property and physiological function, and when the azelaic acid of the present invention is added to general food, the physical property and physiological function of the general food will be improved, and in the present invention, such food with the improved functions is defined overall as “functional food.”

The functional food of the present invention may be used in various applications such as drugs, food, and drinks for preventing or improving a mitochondrial dysfunction-associated disease by regulating mitochondrial biogenesis and degradation in cells to maintain or improve the mitochondrial function, and increasing the number of mitochondria. There is no particular limitation to a type of food. Examples of food to which the material can be added include meats, sausages, breads, chocolate, candies, snacks, cookies, pizza, ramen, other types of noodles, gums, dairy products including ice creams, various types of soups, beverages, teas, drinks, alcohol drinks and vitamin complexes, and in a common sense, all types of food.

A health drink composition according to the present invention may contain various flavoring agents or natural carbohydrates as additional components like a conventional drink. The above-mentioned natural carbohydrates may include monosaccharides such as glucose and fructose, disaccharides such as maltose and sucrose, and polysaccharides such as dextrin and cyclodextrin, and sugar alcohols such as xylitol, sorbitol, erythritol, etc. As sweeteners, natural sweeteners such as thaumatin and a stevia extract, and synthetic sweeteners such as saccharin and aspartame may be used. A proportion of the natural carbohydrates is generally about 0.01 to 20 g, and preferably about 5 to 12 g per 100 mL of the composition of the present invention.

In addition to the components, the composition of the present invention may contain a variety of nutrients, vitamins, minerals (electrolytes), flavoring agents, pectic acid and a salt thereof, alginic acid and a salt thereof, organic acids, protective colloid thickening agents, pH modifiers, stabilizers, preservatives, glycerin, alcohol, or carbonating agents used in carbonated beverages. In addition, the composition according to the present invention may contain flesh for preparing natural fruit juices and vegetable juices. Such ingredients may be used independently or in combination. A ratio of such additive is not particularly limited, but generally selected in a range of 0.01 to 0.20 parts by weight with respect to 100 parts by weight of the composition of the present invention.

The present invention may have various modifications and embodiments, and thus the present invention will be described in further detail below. However, the present invention is not limited to specific embodiments, and it should be understood that the present invention includes all modifications, equivalents and alternatives included in the technical idea and scope of the present invention. To explain the present invention, if it is determined that a detailed description of the related art may obscure the gist of the present invention, the detailed description thereof will be omitted.

Hereinafter, to help in understanding the present invention, exemplary examples will be suggested. However, the following examples are merely provided to more easily understand the present invention, and not to limit the present invention.

### EXAMPLES

### Example 1. Evaluation of Cytotoxicity of Azelaic Acid

To evaluate the cytotoxicity of azelaic acid, an MTT assay was performed. The MTT assay is a test method utilizing the ability of mitochondria to reduce MTT tetrazolium, which is a yellow water-soluble substrate, to non-water-soluble MTT formazan showing a blue-violet color through the action of a dehydrogenase. The MTT reagent was prepared by being diluted in phosphate buffered saline (PBS) at a concentration of 2 to 5 mg/mL. Hepa1c1c-7 cells used in this experiment were purchased from the Korean Cell Line Bank, and cultured in a minimum essential medium Eagle alpha modification medium (MEM-alpha, Hyclone) supplemented with 10% FBS and 1% PEST. For this experiment, the Hepa1c1c-7 cells (4×10⁴ cell/mL) were seeded into a 96-well plate and incubated at 37° C. under 5% CO₂ for 24 hours, and then incubated for 24 hours by adding azelaic acid in a concentration range of 0 to 500 μM. Afterward, each sample was treated with 100 μL of the MTT reagent (4 mg/mL), incubated at 37° C. under 5% CO₂ for 4 hours and treated with 100 μL of dimethyl sulfoxide (DMSO), followed by measuring absorbance at 540 nm. An absorbance level is obtained by quantifying a cell death effect caused by toxicity using a principle in which the absorbance is proportional to a cell count.

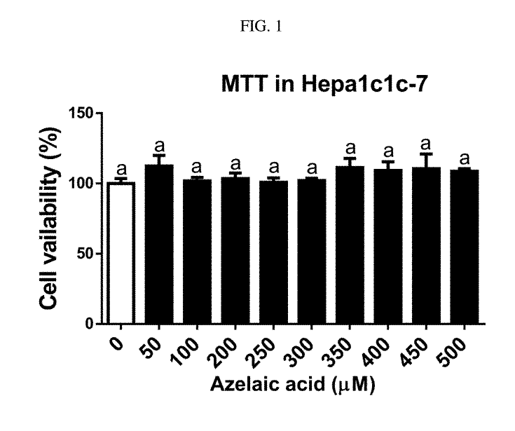

Accordingly, as shown in FIG. 1, it was confirmed that, when the Hepa1c1c-7 cells were treated with 0 to 500 μM of azelaic acid, toxicity was not exhibited at any concentration compared to the control group.

### Example 2. Confirmation of Olfr544 Expression in Muscle Cells and Murine Muscle

2-1. Cell Culture

Murine skeletal muscle cells, C2C12 cells, were purchased from the Korean Cell Line Bank. The cells were cultured using a Dulbecco's modified Eagle's medium (DMEM) supplemented with 20% fetal bovine serum (FBS; HyClone, USA) and 1% antibiotics (penicillin/streptomycin). When the cells reached 50% confluency (the degree of proliferation) under 5% CO₂ (v/v) at 37° C., the cells were subcultured and maintained.

To differentiate the cells, the medium was replaced with DMEM containing 2% horse serum (HyClone), and after 7 days of differentiation, the cells were used in the experiment.

2-2. RT-PCR

RNA extracted from the murine skeletal muscle cells, C2C12 cells, was used to synthesize cDNA using the ReverTra Ace® qPCR RT Kit (TOYOBO, Osaka, Japan). RNA was pre-heated at 65° C. for 5 minutes to increase reaction efficiency, and immediately stored on ice. Afterward, a total of 8 μL of a reagent consisting of 2 μl of 4×DN Master Mix including a gDNA remover, 0.5 μg of mRNA, and nuclease-free water was prepared, and amplification was carried out at 37° C. for 5 minutes. After the reaction, a 5×RT master mix was added to the reagent, and amplification was carried out at 37° C. for 15 minutes and 50° C. for 5 minutes, and 98° C. for 5 minutes, thereby synthesizing cDNA. The resulting PCR product was subjected to electrophoresis on an agarose gel, followed by imaging the gel using ChemiDoc. As a control, L32 was used, and as a positive control, an Olfr544 plasmid was used.

2-3. Analysis of Results

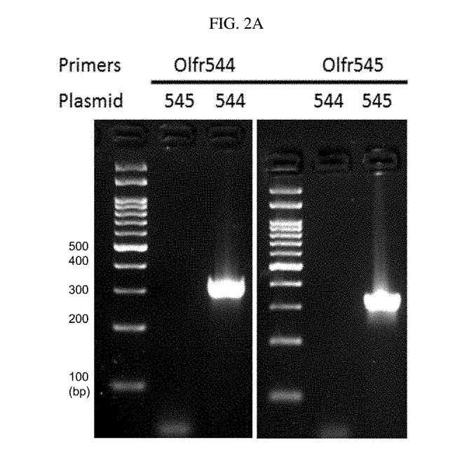

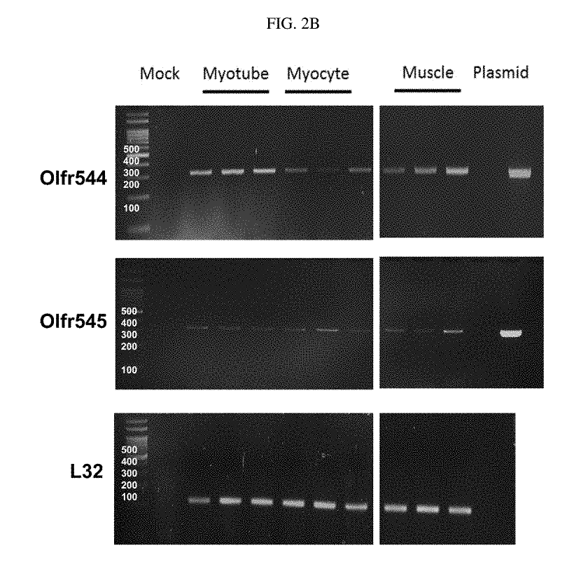

The results of RT-PCR performed to confirm whether Olfr544 is expressed in muscle are shown in FIG. 2A and FIG. 2B.

As shown in FIG. 2B, the expression of an Olfr544 gene was confirmed in myotubes and myocytes, and also confirmed in murine muscle tissue. Subsequently, as an olfactory receptor expressed in muscle cells was reconfirmed using Olfr545 having a similar gene sequence to Olfr544 (see FIG. 2A), it was confirmed that Olfr544 was expressed in muscle.

Based on the above results, the mitochondrial biogenesis efficiency according to the azelaic acid treatment in muscle cells and murine muscle tissue was demonstrated.

### Example 3. Confirmation of the Effect of Promoting Mitochondrial Biogenesis According to Azelaic Acid Treatment in Muscle Cells

3-1. Cell Culture and Treatment with Materials

C2C12 cells differentiated for 7 days as described in Example 2-1 were treated with 12.5, 25 or 50 μM of azelaic acid, and after 24 hours, RNA and proteins were extracted according to the following method and used in the experiment. For a control, instead of azelaic acid, the same amount of DMSO was treated.

3-2. Quantitative Real-Time RT-PCR

qPCR was performed to synthesize cDNA using RNA extracted from cells grown in a culture medium and then treated with 300 μL of the RNAiso plus reagent per well. Subsequently, the synthesized cDNA was subjected to qPCR using the Thunderbird TMSYBR® qPCR Mix reagent (Takara Bio Inc., Japan). A gene expression level was analyzed using the iQ5 Cycler System (Bio-Rad, USA).

3-3. Immunoblotting

Protein extraction was performed using an RIPA buffer (10 mM Tris-HCl, pH 7.5, 1% NP-40, 0.1% sodium deoxycholate, 0.1% SDS, 150 mM NaCl, and 1 mM EDTA), a Halt™ protease and a phosphatase inhibitor reagent (Thermo, USA). As primary antibodies, anti-α-tubulin (1:1000) and anti-PGC-1a (1:500; Santa Cruz Biotechnology, Santa Cruz, USA) were used. The resulting immunoblotting image was analyzed using the ChemiDoc™ touch imaging system and Image Lab 5.2 software (Bio-Rad, PA, USA).

3-4. MitoTracker

After treatment with azelaic acid, the change in mitochondrial density was detected using MitoTracker. C2C12 cells were treated with azelaic acid for 24 hours, and then washed with PBS. Subsequently, a medium containing 200 mM of MitoTracker, i.e., a green probe, was added thereto, and the cells were cultured for 30 minutes. The mitochondrial density was measured by quantifying wavelengths of a part binding to the tracker, which absorbs a wavelength at 490 nm and emits a wavelength at 516 nm, using SpectraMax. Image analysis was performed using a confocal microscope and Zeiss LSM700 version 3.2 software (Carl Zeiss, Germany).

3-5. Analysis of Results

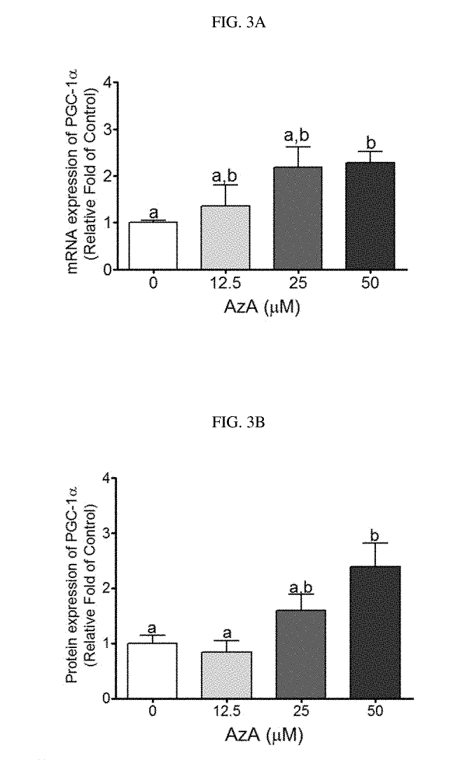

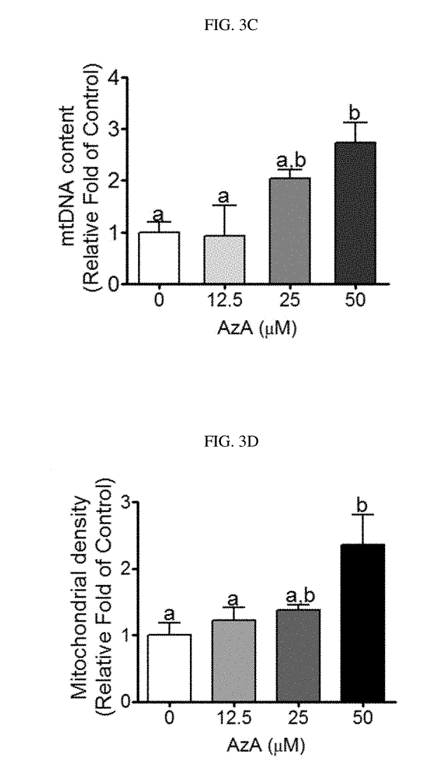

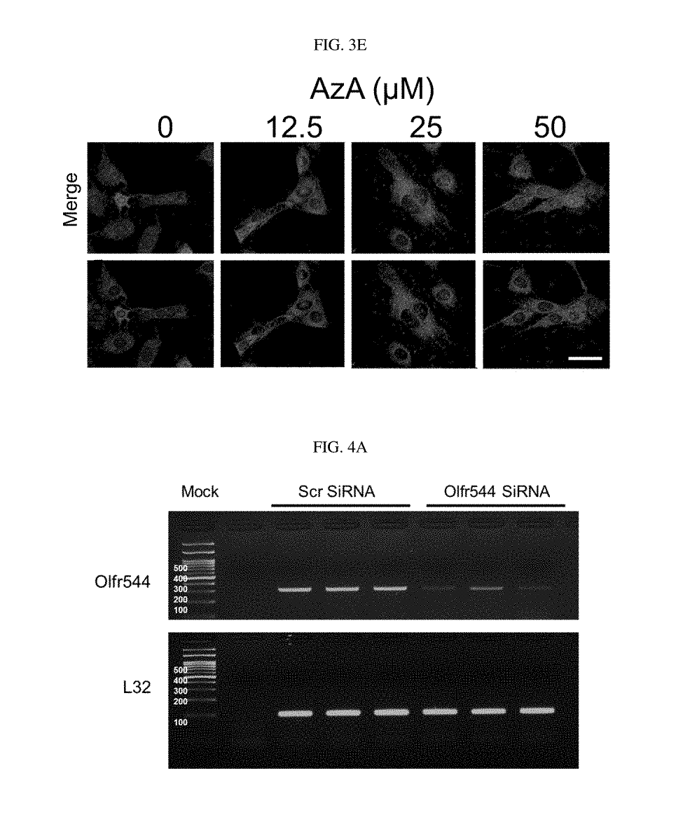

As shown in FIG. 3A and FIG. 3B, when C2C12 cells were treated with azelaic acid, PGC-1a gene and protein expression were increased in the 50 μM-treated group, compared to the control group. Afterward, an mtDNA level and mitochondrial density were analyzed. As a result, it was confirmed that, in the 50 μM azelaic acid-treated group, the mtDNA level was increased about 2.5-fold higher than the control, and the mitochondrial density was significantly increased (see FIG. 3C and FIG. 3D). According to the image analysis using a confocal microscope, in the azelaic acid-treated group, compared with the control group, intensive fluorescence was observed (see FIG. 3E). According to the above results, the treatment of a muscle cell line with azelaic acid increased a major factor in regulating mitochondrial function, PGC-1α, and increased mitochondrial biogenesis, confirming that azelaic acid is effective in increasing mitochondrial biogenesis.

### Example 4. Confirmation of Azelaic Acid Effect of Increasing Olfr544-Dependent Mitochondrial Biogenesis

4-1. siRNA Transfection

The differentiated skeletal muscle cells, C2C12 cells, were transfected with 200 pmol of scramble (control) or Olfr544 siRNA (Santa Cruz, USA) using the Lipofectamine 2000 reagent (Invitrogen, USA) for 6 hours. Subsequently, another transfection was performed for 10 to 12 hours. The transfected cells were used for RNA or protein extraction.

4-2. Quantitative Real-Time RT-PCR

qPCR was performed to synthesize cDNA using RNA extracted from cells grown in a culture medium and then treated with 300 μL of the RNAiso plus reagent per well. Subsequently, the synthesized cDNA was subjected to qPCR using the Thunderbird TMSYBR® qPCR Mix reagent (Takara Bio Inc., Japan). A gene expression level was analyzed using the iQ5 Cycler System (Bio-Rad, USA).

4-3. Immunoblotting

Protein extraction was performed using an RIPA buffer (10 mM Tris-HCl, pH 7.5, 1% NP-40, 0.1% sodium deoxycholate, 0.1% SDS, 150 mM NaCl, and 1 mM EDTA), a Halt™ protease and a phosphatase inhibitor reagent (Thermo, USA). As primary antibodies, anti-α-tubulin (1:1000) and anti-PGC-1α (1:500; Santa Cruz Biotechnology, Santa Cruz, USA) were used. The resulting immunoblotting image was analyzed using the ChemiDoc™ touch imaging system and Image Lab 5.2 software (Bio-Rad, PA, USA).

4-4. Confirmation of Increased mtDNA Expression

The differentiated C2C12 cells were treated with azelaic acid, treated with 300 μL of a lysis buffer (20 mM EDTA, 100 mM Tris, 200 mM NaCl, 0.2% Triton X-100, 100 μg/mL) per well, and cultured at 37° C. for 90 minutes, and the extracted supernatant was treated with the same amount of isopeopanol and 25 μL of a 4M NaCl solution. After storage at −20° C. overnight, distilled water (D.W) was added to a pellet obtained by centrifugation (14,000 rpm, RT, 20 min), thereby extracting mtDNA. Afterward, 2 μl of DNA, 10 μl of the SYBR green PCR master mix, 0.5 μL of each of a forward primer and a reverse primer having a concentration of 10 pmol, and 6.6 μL of sterile distilled water were mixed, and qPCR was performed using the Thunderbird TMSYBR® qPCR Mix reagent (Takara Bio Inc., Japan). A gene expression level was analyzed using the iQ5 Cycler System (Bio-Rad, USA).

4-5. MitoTracker

The change in mitochondrial density was detected using MitoTracker. Olfr544 siRNA-transfected cells were treated with azelaic acid at a concentration of 50 μM for 24 hours, and then washed with PBS. Afterward, a medium containing 200 nM of the MitoTracker green probe was added to the cells, and the cells were cultured for 30 minutes. The mitochondrial density was measured by quantifying wavelengths of a part binding to the tracker, which absorbs a wavelength at 490 nm and emits a wavelength at 516 nm, using SpectraMax.

4-6. Analysis of Results

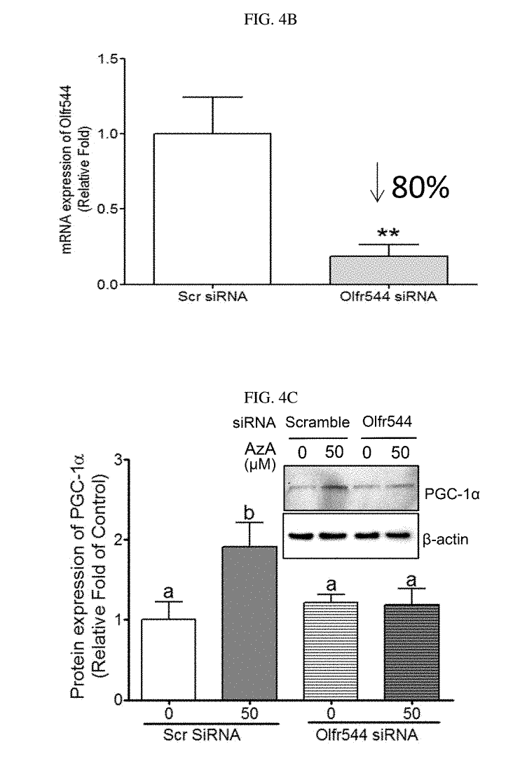

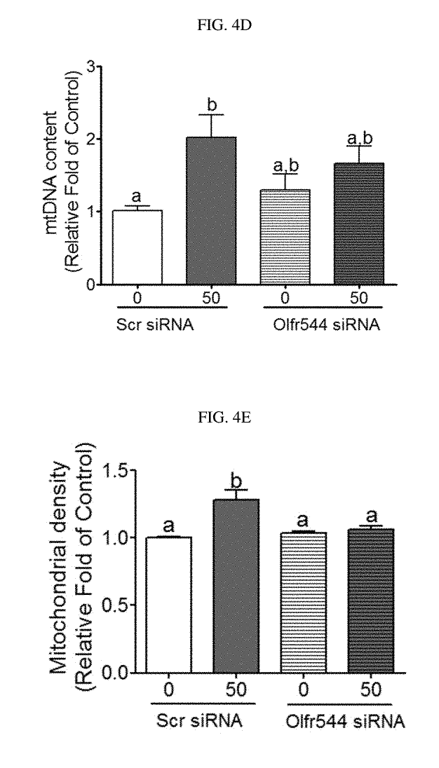

The experimental results are shown in FIG. 4A to FIG. 4E. As a result of confirming Olfr544 expression in normal cells and Olfr544 knockdown cells by RT-PCR, it was confirmed that, compared to a Scr siRNA-transfected control, Olfr544 expression was decreased by about 80% in Olfr544 knockdown cells (see FIG. 4A and FIG. 4B), and then an experiment was carried out as follows. As a result of confirming the PGC-1a protein expression according to azelaic acid treatment in the normal cells and the Olfr544 knockdown cells, it was confirmed that treatment of Scr siRNA-transfected normal cells with 50 μM of azelaic acid significantly increases a PGC-1a protein (see FIG. 4C). It was confirmed that this effect is not shown in the Olfr544 knockdown cells. Afterward, in an experiment of confirming mtDNA and mitochondrial density, although the Olfr544 knockdown cells were treated with azelaic acid, there were no changes in mtDNA and mitochondrial density, but mtDNA was increased about 1.6-fold in the azelaic acid-treated normal cells (see FIG. 4D), and the mitochondrial density was also increased therein (see FIG. 4E). The result demonstrated that azelaic acid induced mitochondrial biogenesis in an Olfr544-dependent manner.

### Example 5. Identification of the Mechanism of Promoting Mitochondrial Biogenesis Through Olfr544 Activation Induced by Azelaic Acid

Examples 3 and 4 showed that Olfr544 activity induced by azelaic acid promotes mitochondrial biogenesis, and subsequently, in this example, it was attempted to confirm by what mechanism does azelaic acid exhibit the above-described effect.

The PGC-1α gene is regulated by a CRE promoter, and CREB activation leads to upregulation of PGC-1α translation. The following experiment was carried out on the premise that Olfr544 activated by azelaic acid promotes PKA-CREB signaling, suggesting that azelaic acid induces PGC-1α expression in skeletal muscle cells.

5-1. siRNA Transfection

The differentiated skeletal muscle cells, C2C12 cells, were transfected with 200 pmol of scramble (control) or Olfr544 siRNA (Santa Cruz, USA) using the Lipofectamine 2000 reagent (Invitrogen, USA) for 6 hours. Subsequently, one more transfection was performed for 10 to 12 hours. After 24 hours, the transfected cells were treated with azelaic acid and used for protein extraction.

5-2. Immunoblotting

Protein extraction was performed using an RIPA buffer (10 mM Tris-HCl, pH 7.5, 1% NP-40, 0.1% sodium deoxycholate, 0.1% SDS, 150 mM NaCl, and 1 mM EDTA), a Halt™ protease and a phosphatase inhibitor reagent (Thermo, USA). As primary antibodies, anti-CREB (1:250), anti-p-CREB (Ser133; 1:500), anti-β-actin (1:1000), anti-α-tubulin (1:1000), anti-ERK1/2 (1:500), anti-p-ERK1/2 (Thr53,54, 1:500), and anti-PGC-1α (1:500; Santa Cruz Biotechnology, Santa Cruz, USA) were used. In addition, anti-LC3B (1:500) was purchased from Novus Biologicals (Novus Biologicals, USA). The resulting immunoblotting image was analyzed using the ChemiDoc™ touch imaging system and Image Lab 5.2 software (Bio-Rad, PA, USA).

5-3. Analysis of Results

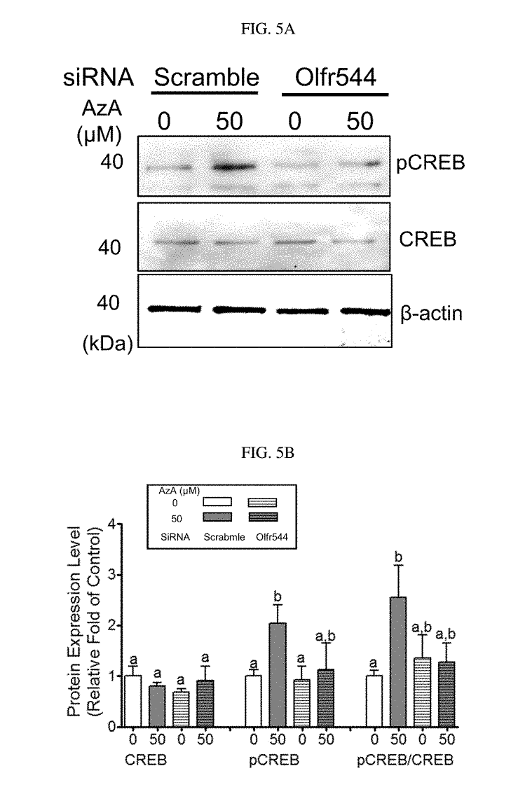

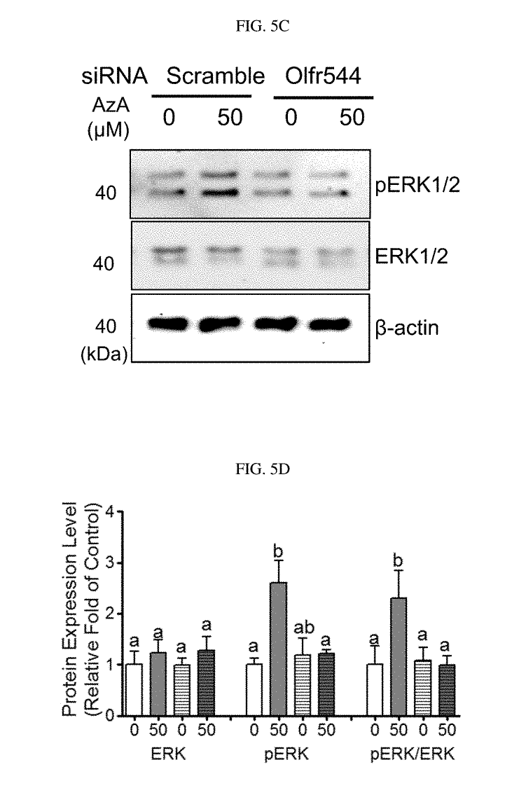

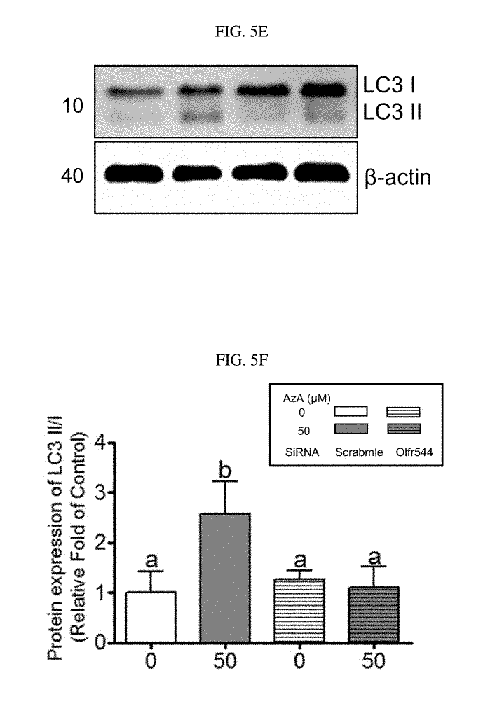

The experimental results are shown in FIG. 5A to FIG. 5F. After Olfr544 knockdown cells and normal cells were treated with azelaic acid, as a result of confirming a CREB activation effect by the expression of a pCREB protein, it was confirmed that the pCREB expression was increased about 2-fold after the normal cells were treated with azelaic acid, but there was no change in pCREB expression in the Olfr544 knockdown cells even by the azelaic acid treatment (see FIG. 5A and FIG. 5B). In addition, it can be confirmed that the expression of a phosphorylated ERK1/2 protein was increased 1.5-fold in skeletal muscle cells (see FIG. 5C and FIG. 5D). Since ERK1/2 phosphorylation is known to enhance autophagy, the expression of an autophagy marker, i.e., the LC3II/LC3I protein, was confirmed. As a result, the azelaic acid treatment induced an about 3-fold increase in expression of the LC3II/LC3I protein, compared to the control group, and this effect disappeared in the Olfr544 knockdown cells (see FIG. 5E and FIG. 5F). Autophagy prevents mitochondrial biogenesis and mitochondrial damage during exercise, and thus plays a pivotal role in the adaptation and ability of muscle tissue with respect to prolonged exercise.

According to the above results, it can be seen that the Olfr544 activity induced by azelaic acid promotes CREB-PGC-1α signaling and ERK1/2 activity in skeletal muscle tissue, thereby inducing autophagy in the skeletal muscle cells.

### Example 6. Confirmation of the Effect of Improving Mitochondrial Biogenesis According to the Administration of Azelaic Acid to Mouse

It was reported that obesity reduces mitochondrial replication and function in skeletal muscle involved in cell oxidation stress, lipotoxicity, and diabetic conditions. Therefore, in this example, it was attempted to analyze a beneficial effect of administration of azelaic acid to the muscle tissue of a mouse in which obesity was induced by HFD.

6-1. Models for Animal Experiments

8-week-old ICR male rats and C57BL/6J were purchased from Samtako (Gyeonggi-do, Seoul), and Olfr544 knockout mice from which exon 2 (161-428 bp) of an Olfr544 gene was removed using a CRISPR-Cas9 system were purchased from Macrogen (Seoul, Korea). All animal experiments were carried out in accordance with the experimental method (Protocol No. KUIACUC-2016-97) approved by the Animal Experiment Ethics Committee at Korea University. In the breeding environment, a 12-hour light cycle, a room temperature of 25 to 31° C., and relative humidity of 60% to 60% were maintained. The mice were freely fed a 60% high-fat diet, and randomly divided into 4 groups (n=7, two groups for normal mice, and the other two groups for knockout (KO) mice). For exact Olfr544 activation, the mice were fasted overnight, and then intraperitoneally injected with azelaic acid (100 mg/kg of body weight). For a control, the same amount of PBS was intraperitoneally injected. In an experiment for long-term administration of azelaic acid, azelaic acid was orally administered at a dose of 100 mg/kg of body weight. During the last week of the experimental period (6 weeks), the mice fasted for 16 hours were anesthetized and sacrificed. The collected muscle tissue was put into a freezing tube, immediately put into liquid nitrogen, and then stored at −80° C.

6-2. Quantitative Real-Time RT-PCR

cDNA was synthesized using RNA extracted by adding the RNAiso plus reagent to murine muscle cells. Subsequently, the synthesized cDNA was subjected to qPCR using the Thunderbird TMSYBR® qPCR Mix reagent (Takara Bio Inc., Japan). A gene expression level was analyzed using the iQ5 Cycler System (Bio-Rad, USA).

6-3. Confirmation of Increased Expression of mtDNA

Murine muscle cells were treated with a lysis buffer (20 mM EDTA, 100 mM Tris, 200 mM NaCl, 0.2% Triton X-100, 100 μg/mL), and cultured at 37° C. for 90 minutes, and the extracted supernatant was treated with the same amount of isopeopanol and 25 μL of a 4M NaCl solution. After storage at −20° C. overnight, distilled water (D.W) was added to a pellet obtained by centrifugation (14,000 rpm, RT, 20 min), thereby extracting mtDNA. Afterward, 2 μl of DNA, 10 μl of the SYBR green PCR master mix, 0.5 μL of each of a forward primer and a reverse primer having a concentration of 10 pmol, and 6.6 μL of sterile distilled water were mixed, and qPCR was performed using the Thunderbird TMSYBR® qPCR Mix reagent (Takara Bio Inc., Japan). A gene expression level was analyzed using the iQ5 Cycler System (Bio-Rad, USA).

6-4. Analysis of Results

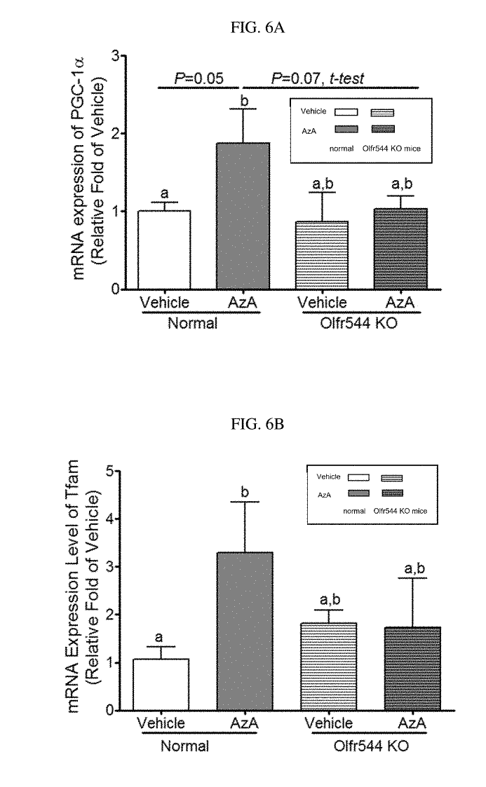

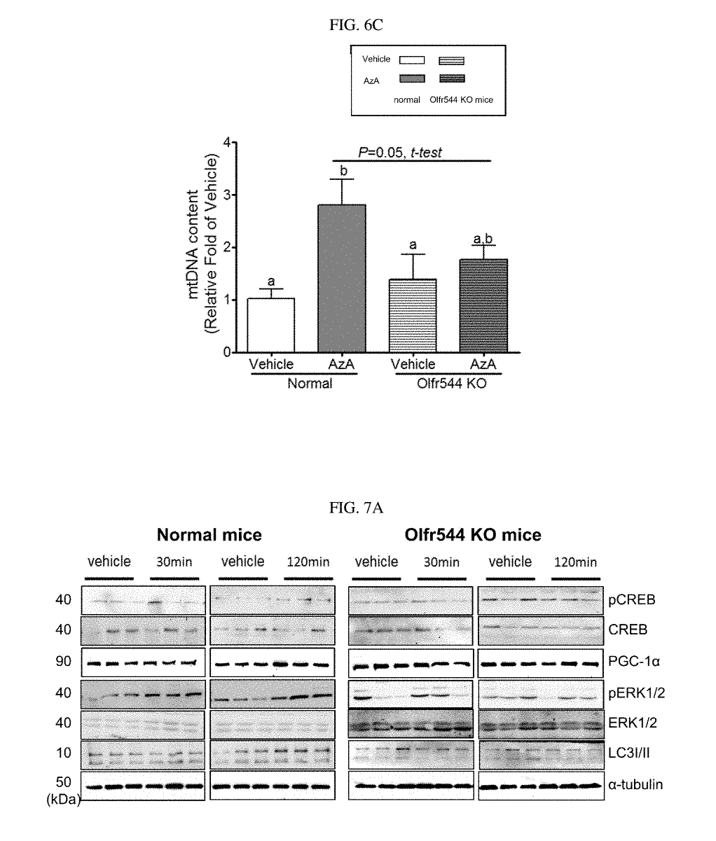

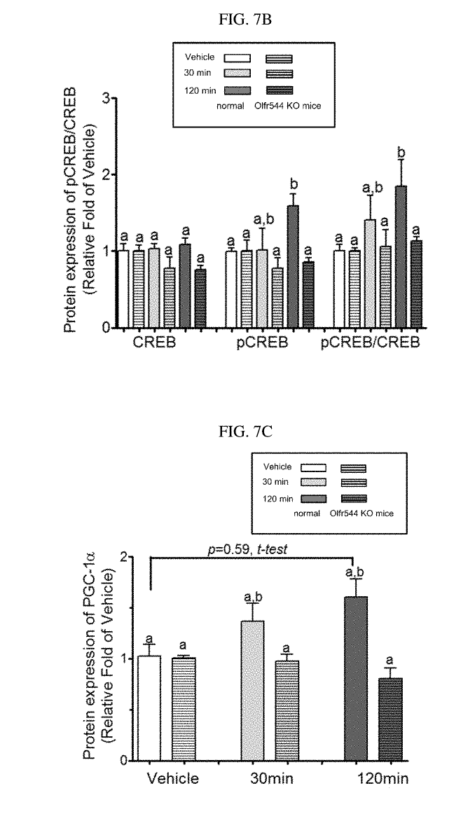

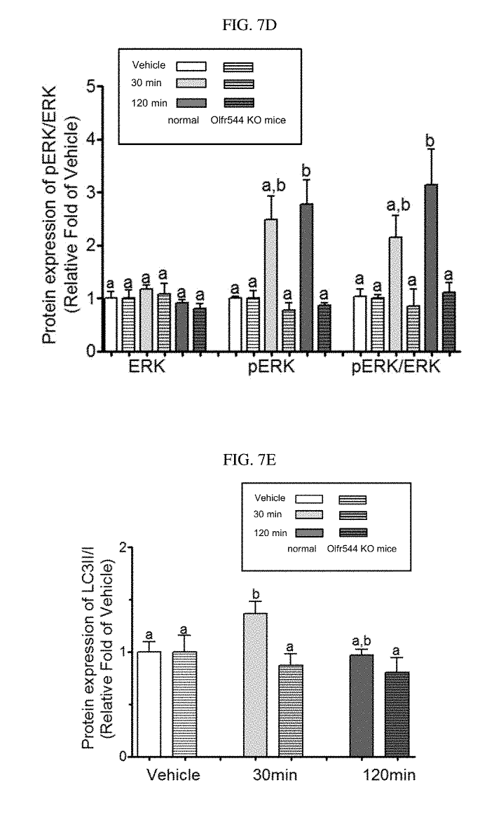

The results of oral administration of azelaic acid are shown in FIG. 6A to FIG. 6C, and the intraperitoneal administration results are shown in FIG. 7A to FIG. 7E.

First, as a result of the oral administration of azelaic acid, for 6 weeks, to the mice in which obesity was induced by HFD, as shown in FIG. 6A, body weight, blood sugar, triglycerides, and sugar tolerance were increased, and PGC-1α mRNA expression was increased about 1.9-fold in the azelaic acid-administered group, compared to the control group. In contrast, there were no changes in the Olfr544 knockout mice even by the azelaic acid administration. In addition, Tfa gene expression involved in mitochondrial PGC-1a downstream signaling was increased about 3.3-fold in normal mice due to azelaic acid (see FIG. 6B). In the knockout mice, there was no change. In addition, in normal mice groups, the azelaic acid-administered mice, compared to the non-administered mice, were increased in mtDNA, but there was no change in the Olfr544 knockout mouse group (see FIG. 6C).

Afterward, as a result of the intraperitoneal injection of azelaic acid, as shown in FIG. 7A to FIG. 7E, 30 minutes and 120 minutes after the intraperitoneal injection, the protein expression of pERK/ERK was compared with the control, confirming that the protein expression of pERK/ERK was increased 2.5-fold/2.8-fold 30 minutes/120 minutes after the injection, respectively. The protein expression of LC3II/LC3I was increased 1.5-fold 30 minutes after the azelaic acid injection, but was not changed 120 minutes after the azelaic acid injection. In the Okfr544 knockout mice, there was no change regardless of the azelaic acid injection. This result shows that the Olfr544 activation by azelaic acid induces autophagy in muscle tissue.

### Example 7. Statistical Analysis

For statistical analysis, a significance test between two groups was performed using a Student's t-test, and the values were *P<0.05, **P<0.01 and ***P<0.001, compared to the control group. The error bars of each graph are expressed as mean±SEM. The significance test between two or more groups used one-way ANOVA, and the error bars of each graph are expressed as mean±SEM.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

1. A method for promoting mitochondrial biogenesis, which comprises administering a composition comprising azelaic acid as an active ingredient to a subject.

2. The method according to claim 1, wherein the method activates Olfactory receptor 544 (Olfr544).

3. The method according to claim 1, wherein the method increases the expression of peroxisome proliferator-activated receptor gamma coactivator 1 alpha (PGC-1α).

4. A method for reinforcing muscle function or preventing muscle aging, comprising:
administering a composition comprising azelaic acid as an active ingredient to a subject,
wherein the reinforcement of muscle function and prevention of muscle aging are accomplished by the activation of mitochondrial function.

5. The method according to claim 4, wherein the method induces the autophagy of mitochondria in cells.

6. The method according to claim 4, wherein the method promotes mitochondrial biogenesis in cells.
